(19) United States
(12) Patent Application Publication
(10) Pub. No.: US 2004/0110731 A1
(43) Pub. Date: Jun. 10, 2004

(54) Photodynamic therapy for the treatment of non-melanoma skin cancer

(76) Inventors: Agnes H. Chan, Coquitlam (CA); Herma C. Neyndorff, Vancouver (CA)

Correspondence Address: Kawai Lau, Morrison & Foerster LLP, Suite 500, 3811 Valley Centre Drive, San Diego, CA, 92130-2332, US

(21) Appl. No.: 10/310,668

(22) Filed: Dec. 4, 2002

(51) Int. Cl. A61K 31/56

(52) U.S. Cl. 514/169

(57) ABSTRACT

The present invention relates to a method of treating non-melanoma skin cancer comprising: (a) intravenously administering a porphyrin based photosensitizer to a subject with non-melanoma skin cancer; (b) irradiating one or more tumors and a circumferential peritumoral margin of at least 1 mm with a light at a wavelength absorbed by said photosensitzer; wherein the irradiating step delivers a light dose of 180 J/cm2. It has been found that the above treatment gives extremely good efficacy while having a surprisingly good cosmetic outcome.

### FIELD OF THE INVENTION

This invention relates to the use of porphyrin based photodynamic therapy (PDT) in the treatment of human disease. The use of PDT and appropriate photosensitizers for treating non-melanoma skin cancer (NMSC) is contemplated and disclosed.

### BACKGROUND OF THE INVENTION

Non-melanoma skin cancers are among the most common of malignant diseases accounting for one-third of all cancers. There are approximately three million cases of NMSC reported annually in North America, Europe, Japan, Australia and South Africa. Basal Cell Carcinomas (BCC) account for 75-80% of all non-melanoma skin cancers. BCC arises from the basal cells of the epidermis and its appendages. It is characterised by slow local growth which is capable of causing extensive tissue damage resulting in loss of organ function and disfigurement. The most common etiological factor in BCC is exposure to ultraviolet light (UV). Consequently, areas of skin with high levels of UV exposure, such as the head or neck, are most commonly affected. Although metastases are rare these neoplasms can be fatal if left untreated. A number of factors can lead to the development of multiple basal cell carcinoma (MBCC) including conditions such as nevoid basal cell carcinoma syndrome (NBCCS, also referred to as basal cell nevoid syndrome (BCNS) and Gorlin-Goltz syndrome), xeroderma pigmentosum, immunosuppression due to intensive immunosuppressive therapy administered after organ transplant, or radiation exposure at a young age particularly for the treatment of acne.

Squamous Cell Carcinomas (SCC) represent the remaining 20-25% of NMSC and are usually fast-growing and prone to metastasize. SCC also tends to affect areas of skin with high levels of UV exposure. SCC is more common in persons who have undergone immunosuppressive therapy. There is some evidence that actinic keratosis (AK) is a first warning sign in the development of SCC. Without treatment, some patients with AK are prone to develop one or more lesions that invade the dermis as squamous cell carcinoma.

Current therapies for NMSC include surgical excision, Mohs' micrographic surgery, cyrosurgery, electrodessication and curettage (ED&C), radiation, and carbon dioxide laser treatment. The cure rates vary according to the therapy, tumor size, and anatomic location but are generally quite high for primary tumors and slightly less for recurrent tumors. However, the current therapies can be time consuming especially where multiple tumors must be treated. Also, the efficacy is highly dependent on the skill of the surgeon and their adherence to the surgical protocol. In addition, there is the possibility of adverse cosmetic effects such as scarring or hypopigmentation. The necessity of treating multiple tumors, such as in MBCC, increases the likelihood of the patient experiencing unacceptable disfigurement or scarring. Tumors on the head, face, or neck present a particular challenge since any disfigurement is likely to have a profound psychological effect. Therefore, treatments for NMSC should be efficacious while having a regard for the appearance of the patient (i.e. acceptable cosmesis).

Photodynamic therapy (PDT) has been used in diverse fields of medical treatment. Generally, PDT involves the delivery of a photosensitizer (PS) to the target tissue and, subsequently, irradiating the target area with light of an appropriate wavelength to activate the PS. This activation results in an agent that modifies or destroys the target tissues. The efficacy of PDT is mediated by the amount of photosensitizing agent taken up by the target tissue and the amount of light energy delivered to the tissue. In general, the higher the dose of photosensitizing and the higher the light dose, the more target tissue is destroyed/modified and, consequently, the more efficacious the treatment. However, in general the increased amount of cell and tissue damage caused by higher drug and light doses lead to less acceptable cosmetic outcomes (or poor cosmesis). Therefore, when treating diseases with PDT the physician must balance the increase in efficacy obtained by higher dosages with the negatives in terms of cosmetic outcome. This is of particular importance when the target tissue is located at psychologically sensitive areas such as the head, face, or neck.

PDT has been proposed as a possible treatment for NMSC. See, for example, “Photodynamic Therapy of Malignancies” James S. McCaughan Jr., pp 52-82 (R. G. Landes Company, Austin Tex., USA, 1992). Recent studies have examined the use of benzoporphyrin derivatives (BPD) as a photosensitizing agent for PDT of NMSC (Seminars in Oncology, Vol. 21, No 6, Suppl 15 (December), 1994: pp 11-14). A benzoporphyrin derivative monoacid A ring (BPD-MA), known as Verteporfin, is currently approved for use in the photodynamic treatment of patients with a variety of ocular diseases characterised by neovasculature. It has been proposed that PDT of NMSC with verteporfin may provide tumor clearing with good to excellent cosmetic results, particularly at a light fluence of 120 J/cm² (“Photodynamic Therapy of Non-Melanoma Skin Cancers with Verteporfin and Red Light Tumor Response and Cosmetic Outcome”—abstract presented by Dr Harvey Lui at the IPA 8^{th} World Congress of Photodynamic Medicine, Jun. 5-9, 2001). The use of 60 J/cm² was unacceptable because of a relatively poor clinical response while the use of 180 J/cm² was unacceptable because of a relatively poor cosmetic outcome.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention relates to a method of treating non-melanoma skin cancer (NMSC) comprising:

(i) administering a porphyrin based photosensitizer (PS) to a subject with non-melanoma skin cancer;

(ii) irradiating one or more tumors and a circumferential peritumoral margin of more than 1 mm with a light at a wavelength absorbed by the PS;

wherein said irradiating activates the PS.

Preferably, the administration is systemic, such as by intravenous injection, and the PS is a benzoporphyrin derivative. Particularly preferred is the use of benzoporphyrin derivative monoacids A ring (BPD-MA) and a wavelength of about 688 nm (±10 nm). The irradiation preferably delivers a total light dose of between about 150 and about 210 J/cm², most preferably about 180 J/cm².

The subject is preferably human, and NMSC is preferably Basal Cell Carcinomas (BCC), nevoid basal cell carcinoma syndrome (NBCCS or basal cell nevoid syndrome (BCNS) and Gorlin-Goltz syndrome), squamous cell carcinoma (SCC) or combinations thereof. Preferably the NMSC treated in the present method is SCC or MBCC.

The circumferential peritumoral margin is preferably 1-8 mm, although larger margins may also be used.

It has been found that the methods of the present invention provides extremely good efficacy in treating non-melanoma skin cancers while also having a surprisingly good cosmetic outcome. The present method is particularly useful in treating carcinomas of particular areas, such as, but not limited to, the head, face, or neck since the cosmetic outcome is very important because of the psychological impact of scarring in these areas. The availability of good cosmesis permits the invention to not be limited to the above cited areas. The invention may be used in a variety of skin tissues and may advantageously be used to treat multiple tumors in a single session because it permits the treatment of peritumoral tissue.

BPD-MA is used in preferred embodiments of the invention in part because it accumulates in hyperproliferative cells and can be activated by light at a wavelength that penetrates tissues and permits activation of the drug in relatively deep level. Systemic administration of BPC-MA permits multiple tumors to easily be treated in a single session. Indeed, depending on the location of the tumors and the number of available light sources as many as 20 or 30 tumors can be treated in a single session. This is particular advantageous in cases of multiple tumors in a small area or metastasis.

### BRIEF DESCRIPTION OF THE INVENTION

The present method involves intravenous administration of a porphyrin based PS, such as BPD-MA, to a subject with NMSC, such as MBCC or SCC. The tumor(s) and a circumferential peritumoral margin of more than 1, preferably of about 1 to about 8, of about 2 to about 6, or of about 3 to about 4 mm, are then irradiated with light at a wavelength absorbed by the PS. The total light dosage is from about 150 to about 210 J/cm², although dosages of about 180 J/cm² (±36 J/cm²). For a variety of benzoporphyrin derivatives, the wavelength is about 688 nm (±10 nm). These elements and the method are described in more detail below.

As used herein, the term “circumferential peritumoral margin” refers to the area immediately adjacent to, or surrounding, a tumor site but not showing any signs of carcinoma. Since this is apparently healthy tissue it is not a physician's first instinct to irradiate it. However, the efficacy of the present invention is improved when a circumferential peritumoral margin of more than 1 mm, such as, but not limited to, 1-8 mm, 2-6 mm, or 3-4 mm, is treated along with the tumor. Without being bound by theory, and offered solely to improve understanding of the invention, it is believed that treating the surrounding normal (peritumoral) tissue with PDT contributes to the efficacy of the present invention in the treatment of NMSC. It is also believed to be surprising that the treatment of a peritumoral region with PDT does not significantly, and negatively, impact the color, profile, and texture of the skin to result in poor cosmesis. The availability of good cosmesis outcome, combined with a good clinical response, after an extended period of time is a surprising advantageous outcome of the present invention.

The present invention is also advantageously used when multiple tumors are located too closely together to be separated by the required margin. The whole cluster of two or more tumors can be considered as a single tumor and the peritumoral margin calculated accordingly. The whole cluster can be irradiated simultaneously.

As used herein, the term “tumor site” refers to the tumor plus the circumferential peritumoral margin.

Photosensitizing Agent

BPD-MA is a “Green porphyrin” (Gp), which are porphyrin based PS as discussed herein. These are porphyrin derivatives that can be obtained by reacting a porphyrin nucleus with an alkyne in a Diels-Alder type reaction to obtain a mono-hydrobenzoporphyrin. Such resultant macropyrrolic (macrocyclic) compounds are called benzoporphyrin derivatives (BPDs), which is a synthetic chlorin-like porphyrin with various structural analogues, as shown in U.S. Pat. No. 5,171,749. Typically, green porphyrins are tetrapyrrolic porphyrin derivatives obtained by Diels-Alder reactions of acetylene derivatives with protoporphyrin under conditions that promote reaction at only one of the two available conjugated, nonaromatic diene structures present in the protoporphyrin-IX ring systems (rings A and B). The preparation of BPD-MA is described in detail in U.S. Pat. No. 5,095,030, which is hereby incorporated by reference as if fully set forth. BPD-MA is commercially available as verteporfin (available under the tradename Visudyne® from Novartis Ophthalmics, Duluth, Ga. 30097, USA)

Green porphyrins disclosed in U.S. Pat. Nos. 5,283,255, 4,920,143, and 4,883,790 may also be used in the practice of the invention. Green porphyrin derivatives, discussed in U.S. Pat. No. 5,880,145 and related U.S. patent application Ser. No. 09/265,245 may also be used. Other BPDs include BPD-MB, BPD-DA, BPD-DB, as well as the A-ring compounds A-EA6 and A-B3, described in U.S. Pat. Nos. 5,929,105 and 5,880,145.

The porphyrin derivative, such as BPD-MA, active should be formulated in such a way that it is suitable for intravenous administration, although topical formulations may also be used. Any formulation in which the drug can be solubilized and which releases the drug in the plasma can be used herein. Examples of suitable formulations can be found in U.S. Pat. Nos. 5,214,036, 6,074,666, and 5,707,608.

A preferred formulation is described in U.S. Pat. No. 5,707,608 (incorporated herein by reference). This document describes a BPD-MA containing composition that comprises an aqueous lipid complex. Preferably, the lipid complex comprises phospholipids. Preferred phospholipids include phosphatidylglycerols, phosphatidylcholines, or mixtures thereof. Specific phospholipids that may be useful in the lipid complex include, but are not limited to, dimyristoyl phosphatidyl choline, phosphatidyl choline, dipalmitoylphosphatidy choline, distearoyl phosphatidyl choline, soy phosphatidyl choline, egg phosphatidyl glycerol, phosphatidyl ethanolamine, phosphatidic acid, phosphatidylserine, phosphatidylinositol, or mixtures thereof. It is preferred that the lipid complex comprises dimyristoyl phosphatidyl choline, egg phosphatidyl glycerol, or mixtures thereof. It is especially preferred that the lipid complex comprise a mixture of dimyristoyl phosphatidyl choline and egg phosphatidyl glycerol. The lipid complex composition preferably also comprises saccharide. Preferred saccharides for use herein include, but are not limited to, lactose, trehalose, maltose, maltotriose, palatinose, lactulose, sucrose, or mixtures thereof. Polysaccharides may also be used but disaccharides, or mixtures thereof, are preferred.

The method of the present invention preferably involves administration of 12-18 mg of a porphyrin derivative, such as BPD-MA, per m² of subject or patient body surface area (BSA). In a particularly preferred embodiment of the invention, 14 mg/m² of BPD-MA are administered. The BSA can be calculated according to the following equation:

√((height (cm)×weight (kg))÷3600)

The preferred BPD-MA for use in the present method is verteporfin, available under the tradename Visudyne® (Novartis Ophthalmics, Duluth, Ga. 30097, USA). This is a sterile, lyophilized, lipid-based powder that is reconstituted before use. The reconstitution should not be performed in direct bright light. Once reconstituted the solution should be intravenously infused within 4 hours.

Administration of Photosensitizer

The porphyrin derivative, such as BPD-MA, is preferably administered intravenously. The intravenous administration can be performed in any suitable fashion. Preferably, the BPD-MA is administered by controlled intravenous infusion over a time period of about 7 to about 15 minutes. More preferably the BPD-MA is infused over a 10 minute period. Administration over about 30, about 45, about 60, about 75, about 90, about 120, about 150, about 180, about 210, or about 240 minutes may also be used.

Preferably, there is at least a one hour time period between the start of infusion and the administration of the activation energy to the tumor site. Preferably, no additional activation energy is administered after 3 hours from the start of infusion. The change in BPD-MA levels over a 3 hour period has been determined as sufficiently small that a fixed light dose can be used. Using a fixed light dose is more convenient for the physician and there is less chance of error.

The preferred method of administration is:

(i) by controlled IV infusion of verteporfin over a time period of 10 minutes;

(2) waiting one hour from the start of the infusion; and

(3) administering the activation energy to tumor site(s) for a maximum of a further two hours.

Activation Energy

After the administration of the BPD-MA, a light dose of about 150 to about 210, preferably about 180 J/cm² (±36 J/cm²) is delivered to each tumor site. Preferably 180 J/cm² (±18 or 10 J/cm²) is delivered to each tumor site. BPD-MA has a peak absorption at 688 nm; therefore, the activation energy used with it has a wavelength of 688 nm (±10 nm). The peak absorption light frequencies of other porphyrin derivatives are known or readily determined. It is also preferred that the light source provides energy with a full-width half max of 27 nm (±12.5 nm).

Preferably, the light dose is delivered to the tumor site over about a 15 minute period using an irradiance of about 200 mW/cm². Longer or shorter time periods of irradiation can also be used if the irradiance is adjusted down or up, respectively, so that the required light dose is delivered. It has been found that delivering the light over 15 minutes provides an excellent response while minimising the amount of time required to perform the irradiation step.

If the tumor and the circumferential peritumoral margin is smaller than the smallest light treatment field obtainable from the source, the skin surrounding the circumferential peritumoral margin may be optionally masked to limit the peritumoral margin. This can be achieved, for example, by using masking material that does not transmit the light frequency used or absorbs the frequency to prevent its transmission through the material. Non-limiting examples of masking material include aluminum foil or zinc oxide creams.

Activation Energy Source

The light dose herein may be provided by any suitable means. Preferred energy sources are lasers, filtered full spectrum lamps, filtered light from incandescent light sources, light emitting diodes, or combinations thereof. Preferably, the light dose is provided by a laser or light emitting diodes. More preferably the light dose is provided by light emitting diodes.

Preferably, the device comprises multiple light emitting diode emitters mounted in a two-dimensional array so that projected light can be used to illuminate a surface. The light source may be fitted with an aperture that can be used to control the size of the treatment spot.

A suitable light emitting diode device is the QB QUANTAMED 688 available from Quantum Devices Inc., 112 Orbison Street, P.O. Box 100, Bameveld, Wis. 53507, USA.

Article

The present invention also relates to an article or kit comprising a PS and instructions, or a label indicating its use, for its use to treat NMSC. The instructions or label preferably relate to the methods of the present invention, and the PS is preferably BPD-MA. A non-limiting example of instructions comprise a description of intravenously administering the porphyrin, or benzoporphyrin, derivative to a subject with NMSC, and, after at least 1 hour but not more than 3 hours from the start of infusion, irradiating one or more tumors and a circumferential peritumoral margin of 3-4 mm. In the case of BPD-MA, the instructions will indicate a light dose of 180 J/cm² (±36 J/cm²) and a wavelength of 688 nm (±10 nm).

Preferably the BPD-MA is verteporfin. Preferably the instructions indicate that 12-18 mg of BPD-MA are administered per m² of body surface area (BSA) of a human patient afflicted with NMSC. Most preferably, the instructions indicate that 14 mg of BPD-MA are administered per m² of BSA. Preferably, the instructions indicate that the BSA can be calculated by taking the square root of the sum of the height in cm multiplied by the weight in kg and divided by 3600. Preferably the instructions indicate that the infusion should be performed over 10 minutes. Preferably the instructions indicate that the irradiation of each tumor should be performed over 15 minutes. Preferably the instruction indicate that the light source should be a light emitting diode source.

### EXAMPLE 1

Visudyne¹ was diluted to 30 ml with 5% dextrose in water. This solution was then administered intravenously by a ten minute infusion to fifty-four patients having multiple non-melanoma skin tumors. The final concentration of BPD-MA administered was 14 mg per m² of body surface area. The body surface area was calculated by taking the square root of the sum of (height (cm)×weight (kg))÷3600. ¹ available from Movartis Opthalmics, Duluth, Ga. 30097, USA.

One to three hours after the start of drug infusion the patients either had their tumors treated with a light dose of 60 or 120 J/cm² (as comparative examples) or with a light dose of 180 J/cm² according to the invention. The light doses were all of red (688 nm) LED light with a fluence rate of 200 mW/cm². A total of 421 tumors were treated. 194 received 60 J/cm², 117 received 120 J/cm², and 110 received 180 J/cm².

Tumors were examined for clinical response after 3 months. Tumors on six patients from the comparative 60 J/cm² group showed no response after 3 months and were retreated with 18 mg/m² of BPD-MA and 60 J/cm².

The efficacy of the treatment and the cosmetic outcome were assessed at 6 months and 24 months from the start of treatment. As a measure of efficacy, each treatment site was visually assessed by a physician to see if it was tumor free. The percentage of treatment sites that were judged to be free of tumors is shown in Table 1 (Clinical Complete Response). The treatment sites were also visually graded by the physician to assess the Cosmetic Outcome. Each tumor site was graded as an excellent, good, satisfactory, or less than satisfactory based on the color, profile, and texture of each treated site.

The percentage of treatment sites that were judged to be satisfactory or better is shown in Table 1.

TABLE 1

| | 6 MONTHS | | | 24 MONTHS | | |
|---|---|---|---|---|---|---|
| | *60 J/cm² | *120 J/cm² | 180 J/cm² | *60 J/cm² | *120 J/cm² | 180 J/cm² |
| Clinical Complete | 78.3% | 88.5% | 98.0% | 51.3% | 78.6% | 95.4% |
| Response | | | | | | |
| Tumor Cosmetic | 90.6% | 87.5% | 64.8% | 91.8% | 76.4% | 85.5% |
| Outcome | | | | | | |

*comparative example

From the data at 6 months it can be seen that cosmesis suffered with increasing light dose. However, and unexpectedly, the 24 month data show that the light dose (180 J/cm²) with the poorest cosmesis at 6 months did not have a negative long term effect on the cosmetic outcome. Moreover, it was also the most efficacious light dose for a beneficial clinical response. Therefore, the methods of the present invention surprisingly provide both good cosmesis and excellent efficacy.

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

We claim:

1. A method of treating non-melanoma skin cancer comprising:
(a) intravenously administering benzoporphyrin derivative monoacid A ring to a subject with non-melanoma skin cancers;
(b) irradiating one or more tumors and a circumferential peritumoral margin of 1-8 mm with a light at a wavelength of 688 nm (±10 nm);
wherein the irradiating step delivers a light dose of 180 J/cm² (±36 J/cm²).

2. A method of treating non-melanoma skin cancer comprising:
(a) intravenously administering verteporfin to a subject non-melanoma skin cancers;
(b) irradiating one or more tumors and a circumferential peritumoral margin of 1-8 mm with a light at a wavelength of 688 nm (±10 nm);
wherein the irradiating step delivers a light dose of 180 J/cm2 (±36 J/cm2).

3. A method according to claim 1 wherein the circumferential peritumoral margin is 3-4 mm.

4 A method according to claim 1 wherein the non-melanoma skin cancer is multiple basal cell carcinoma.

5. A method according to claim 1 wherein the non-melanoma skin cancer is squamous cell carcinoma.

6. A method according to claim 1 wherein the benzoporphyrin derivative monoacid A ring is delivered in the form of an aqueous lipid complex.

7. A method according to claim 1 wherein of 12-18 mg of BPD-MA is administered per m² of body surface area.

8. A method according to claim 1 wherein the intravenous administration is by an infusion over a 10 minute period.

9. A method according to claim 1 wherein the light dose is provided by light emitting diodes.

10. A method according to claim 1 wherein the light dose is administered over a 15 minute period.

11. A method according to claim 1 wherein the light dose is administered at an irradiance of about 200 mW/cm2.

12. An article comprising:
(a) verteporfin, and
(b) instructions for the use of the verteporfin for treating non-melanoma skin cancer by intravenously administering the verteporfin to a subject with non-melanoma skin cancers, and, after at least 1 hour but not more the 3 hours from the start of infusion, irradiating one or more tumor and a circumferential peritumoral margin of 3-4 mm with a light dose of 180 J/cm2 (±36 J/cm2) said light being of a wavelength of 688 nm (±10 nm).

13. An article according to claim 12 wherein the instructions indicate that 14 mg of BPD-MA should be administered per m2 of body surface area.

14. A method according to claim 2 wherein the circumferential peritumoral margin is 3-4 mm.

15. A method according to claim 2 wherein the non-melanoma skin cancer is multiple basal cell carcinoma.

16. A method according to claim 2 wherein the non-melanoma skin cancer is squamous cell carcinoma.

17. A method according to claim 2 wherein of 12-18 mg of BPD-MA is administered per m² of body surface area.

18. A method according to claim 2 wherein the intravenous administration is by an infusion over a 10 minute period.

19. A method according to claim 2 wherein the light dose is provided by light emitting diodes.

20. A method according to claim 2 wherein the light dose is administered over a 15 minute period.

21. A method according to claim 2 wherein the light dose is administered at an irradiance of about 200 mW/cm².
